SEQUENCE LISTING (18 sequences)

SEQ ID NO: 1 - DNA, 209 bp, cucumis
  BGL Template
tatgatcttcctcaagtcttggaagaagagtataaaggcctattgagtgacagagtagtgaaggattttgcagattatgcagaattttgtttcaaaacgtttggggatagagttaagaattggatgacgtttaacgaaccaagagtcgtggcagctctaggatatgataatggtttttttgctcctgggaggtgttctaaagcatacgg

SEQ ID NO: 2 - DNA, 159 bp, cucumis
  PRP Template
atgccccattgacacgctgaagttgggagcgtgtgtggacttgttgggtgggttgatccatatcggaattggtgaccgtacgaaacaaacttgctgccctgttcttgaaggactagtggatttggatgcggcagtttgtttgtgtaccaccattaaagc

SEQ ID NO: 3 - DNA, 173 bp, cucumis
  RPL12 Template
tcaaagagcccgaacgcgaccgcaagaagaccaagaacatcaagcacaatggtaatatctcgcttgacgatgttattgagattgctagggttatgcgccccaggtctatggctaaggatctcagtggatccgttaaggagattctcggtacttgcgtttctgttgggtgtacg

SEQ ID NO: 4 - DNA, 216 bp, cucumis
  THA Template
ttacccaaaagatgatgcaaccagcacattcacttgccctgggggaaccaactatagggttgttttctgcccttaaaaccagattatatagatataaaaaggaaaccaaacgttacatgaatagttaaagagttgccatatatattatataccttttatataggtatatatatggtgtaatttgtaataagatttggatatggttggtaaatgagc

SEQ ID NO: 5 - DNA, 182 bp, cucumis
  RPL10 Template
cgatgcaaggacagcaacagccagcatgctcaggaggctctccgtcgtgctaagtttaagttccctggtcgtcaaaagatcattgttagcaggaagtggggattcactaaatttagccgagctgattacctcaagttcaagtcagagaacaagattatgccagatggtgttaatgctaagct

SEQ ID NO: 6 - DNA, 17 bp, Artificial Sequence
  RPL10 Sequencing Primer
tcattgttagcaggaag

SEQ ID NO: 7 - DNA, 15 bp, Artificial Sequence
  THA Sequencing Primer
cattcacttgccctg

SEQ ID NO: 8 - DNA, 8 bp, Artificial Sequence
  Subsequence of RPL10 to be analyzed
tggggatt

SEQ ID NO: 9 - DNA, 8 bp, Artificial Sequence
  Subsequnce of THA to be analyzed
ggggaacc

SEQ ID NO: 10 - DNA, 17 bp, Artificial Sequence
  RPL12 Sequencing Primer
aagaacatcaagcacaa

SEQ ID NO: 11 - DNA, 16 bp, Artificial Sequence
  PRP Sequencing Primer
gtgggttgatccatat

SEQ ID NO: 12 - DNA, 8 bp, Artificial Sequence
  Subsequnce of RPL12 to be analyzed
tggtaata

SEQ ID NO: 13 - DNA, 10 bp, Artificial Sequence
  Subsequnce of PRP to be analyzed
cggaattggt

SEQ ID NO: 14 - DNA, 19 bp, Artificial Sequence
  BGL Sequencing Primer
ttgagtgacagagtagtga

SEQ ID NO: 15 - DNA, 10 bp, Artificial Sequence
  Subsequnce of BGL to be analyzed
aggattttgc

SEQ ID NO: 16 - DNA, 87 bp, cucumis
  Complimentary strand of BGL Template
acttgaggaagatcatagaaccttcttctcatatttccggataactcactgtctcatcactyctaaaacgtctaatacgtcttaaaa

SEQ ID NO: 17 - DNA, 12 bp, Artificial Sequence
  Subsequnce of THA to be analyzed
ggggaaccaact

SEQ ID NO: 18 - DNA, 10 bp, Artificial Sequence
  Subsequnce of RPL10 to be analyzed
tggggattca

(19) United States
(12) Patent Application Publication
(10) Pub. No.: US 2004/0229259 A1
(43) Pub. Date: Nov. 18, 2004

(54) Expression profiling

(76) Inventors: Simon Albertus Langeveld, Bennebroek (NL); Dianne Antoinette Maria van der Kop, Wageningen (NL); Anne Douwe de Boer, Dreumel (NL)

Correspondence Address: TRASK BRITT, P.O. BOX 2550, SALT LAKE CITY, UT, 84110, US

(21) Appl. No.: 10/809,824

(22) Filed: Mar. 25, 2004

(30) Foreign Application Priority Data
Sep. 24, 2002 (WO) PCT/NL02/00615
Sep. 25, 2001 (EP) 01203617.4

(51) Int. Cl. C12Q 1/68

(52) U.S. Cl. 435/6

(57) ABSTRACT

The invention relates to DNA sequencing methods useful in expression profiling. The invention provides a semi-automated high-throughput testing facility for expression profiling with immediate use in expression profiling such as in testing plant quality of horticultural and agricultural products.

### PRIORITY CLAIM

This application claims priority to International Application No. PCT/NL02/00615, filed Sep. 24, 2002, and published as PCT International Publication No. WO 03/027324 A2, which application designated the United States.

### TECHNICAL FIELD

The invention relates to methods useful in expression profiling.

### BACKGROUND

Criteria for quality and environmental safety of fresh products are getting more and more strict due to a changing awareness of consumers, trade and agricultural and horticultural industry. A parallel development is the increasing need for quality tests. A clear market exists for objective detection methods that will enable quality ranking. By using such tests, it will become possible to predict quality decay, to certify product batches and to monitor the effect of external treatments. Tests are necessary to monitor quality during handling, transport and storage for food products like potatoes, vegetables and fruit, or horticultural products like trees and shrubs, potted plants and cut flowers. Tests are also necessary to predict the time of harvest or planting for food and horticultural products. In addition, tests are necessary to monitor quality in specialized situations, e.g., to monitor the time of low-temperature treatment to induce flowering in various bulbous species or to monitor the exposure to air pollution in indicator species.

Until recently, quality was not, or only poorly, defined and was usually only judged by visually examining the product, usually based on subjective criteria, that were difficult to quantify. An innovation for the development of objective tests, however, is the use of modern biotechnology. The recent advances in equipment for detection and analysis, combined with novel molecular and biochemical techniques (e.g. functional genomics) to identify and isolate markers that typify a defined physiological stage, definitely provide new opportunities for the development of sensitive tests.

Genomics and bio-informatics rapidly provide a growing number of markers for a large array of products. The development of equipment that is able to use this information for diagnostic purposes becomes increasingly important.

### SUMMARY OF THE INVENTION

The invention provides a semi-automated high-throughput testing facility for expression profiling with immediate use in testing plant quality of horticultural and agricultural products which can be executed in one reaction vessel per profile tested. However, a method according to the invention is also applicable to other organisms where expression profiling may be at order. Expression profiling is in general done by annealing a known but quantitatively to-be-detected marker nucleic acid with a detecting nucleic acid, which most times is provided with a label. Other systems use an array format, wherein the nucleic acid(s) to be detected adhere to an array. Here, we provide a method for expression profiling wherein the nucleic acid(s) to be quantified are at least partly sequenced. Preferably, the test is based on the detection of RNA expression of a set of biomarkers. In principle, this approach is universally applicable, since bio-markers can be used for every quality determination with a physiological background. For every class of problems concerning quality, however, markers must be identified and isolated. With quality loss, e.g., stress-induced senescence, oxidative damage or desiccation, the plant tissues will go through various physiological stages in which different genes are switched on or off. The levels of expression of these marker genes reflect the physiological stage and, therefore, the condition and quality of the plant and/or product. In general, the invention provides a method for determining a developmental or physiological stage of an organism by determining gene expression profiles of the organism or parts (such as cells or cell extracts) thereof, the method at least comprising determining the expression of at least a first gene and a second gene, or fragments thereof, involved in development of the organism, the method comprising a) providing a first (single-stranded) nucleic acid template derived from the first gene and a second (single-stranded) nucleic acid template derived from the second gene b) hybridizing at least one primer to the first template and at least one other primer to the second template and c) determining binding of the primers to the templates in one reaction vessel, whereby the primers essentially not share the same or similar binding sequences and are each directed at a different target gene or gene product representative for each of the various genes or gene products to be detected. The first gene and a second gene not being mere allelic variants of a single gene but being two or more substantially different genes (having less than 70%, and preferably less than 50% nucleotide identity, preferably at least in the region to be amplified). With “reaction vessel,” an entity, such as a droplet or test tube, in which a reaction takes place is meant.

In a preferred embodiment, a method is provided wherein a template comprises RNA. For fresh products, quality usually is a reflection of the physiological condition of the product. There is a direct relation between the pattern of gene expression on the RNA and protein level, and the physiological status of a cell. Therefore, it is possible to isolate marker RNAs that are indicative for the physiological changes and, as a consequence, for the quality of cells, tissues or organisms. RNAs are, for example, extracted from tissues by use of organic solvents and optionally subsequently separated from DNA in a selective precipitation step, for example, including high-molar solutions of LiCl. Reverse transcription of RNA into cDNA is performed by a reverse transcriptase step, such as by using avian myeblastosis virus (AMV), reverse transcriptase using oligodT-mers or gene-specific oligonucleotides to prime the synthesis of cDNA in a reaction with polyA mRNA or total RNA. Amplification of part(s) of any marker gene can subsequently be performed in a polymerase chain reaction (PCR) using template-specific primers to enable detection by the method described below.

It is preferred that the method includes a sequencing reaction that can be initiated by a primer, preferably, the primer can initiate a sequencing reaction carried out by a DNA-polymerase. It is also provided to use a DNA polymerase that is RNA dependent.

In a preferred embodiment, the invention provides a method wherein a dATP or ddATP analogue is used which is capable of acting as a substrate for the polymerase but incapable of acting as a substrate for a pyrophosphate-detection enzyme such as ATP-sulfurylase, such as is known from a method of sequencing DNA generally called pyrosequencing (WO/98/13523). Pyrosequencing is originally developed to sequence large amounts of short to medium length DNA sequences. Using this technique, single nucleotide polymorphisms (SNPs) can be detected in a simple way and in large quantities. SNPs have been developed for use in genetic studies and are currently being implemented in medical diagnostics. In the pyrosequencing reaction, DNA polymerase catalyzes the incorporation of a deoxynucleotide triphosphate, complementary to template nucleotide base, into the new DNA strand, while releasing a pyrophosphate. Via several enzymatic steps, the pyrophosphate is converted into light. The amount of light is proportional to the number of nucleotides incorporated at that position and, therefore, proportional to the amount of matching template. After each reaction, the surplus of substrate is removed. Pyrosequencing therefore results in simultaneous determination of the sequence and quantification of the different SNPs in the template mix. That the method of pyrosequencing is also applicable to quantitative nucleic acid analyses of multiple gene targets such as expression profiling in general comes as a surprise, considering its focus on qualitative nucleic acid detection such as in SNP detection. In the original technology, amplified single-stranded DNA is used as a template for primer annealing. For the detection of RNA markers as provided herein, the technology is modified. Either RNA, first strand cDNA or amplified single-stranded cDNA might be used as a template. If RNA is used as a template, the DNA polymerase is replaced by a (modified) reverse transcriptase.

In an even more preferred embodiment, the invention surprisingly provides the insight that the principle of pyrosequencing, such as, for example, also known from Ronaghi et al., Analytical Biochemistry 242:84-89 (1996), can also be applied to the detection and quantitative analyses of two (or even more) templates at once, especially those templates that are derived from two or more substantially different genes (e.g. less than 70%, and preferably less than 50% identity, at least in the region to be amplified) and thus separately identifiable genes, whereby their relative ratio can be established instead of only from essentially one single gene wherein allelic variation is to be detected, thereby distinguishing itself from WO 01/42496 or Nygren et al. Analytical Biochemistry 288, 28-38 (2000), whereby a single primer is used directed at essentially or substantially the same template, i.e. directed at the target gene and at competitor sequences that differ only in a few nucleotides but are substantially similar to the target gene in the region to be amplified.

Sensitivity is even further increased when the detection and quantitative analyses of the template or templates is achieved by employing in step b) one or more additional primers that are designed to hybridize right in front or at least in close proximity of a relative short (i.e 1 to 10, preferably 2 to 5, most preferably 3 nucleotides long) stretch of identical nucleotides on a template to be detected, which stretches are than sequenced during step c). A stretch does not necessarily have to be located immediately adjacent to the selected primer sequence but can be some nucleotides (i.e. 1 or 2) apart. Considering that the quantitative determination that relies on the detection of a plurality (two or more) of the identical stretches per template which are measured, for example, by pyrophosphate release during the pyrosequencing reaction, detection sensitivity is further increased. Considering that, for example, a codon comprises 3 nucleotides and considering the relative frequency of individual codons in each nucleotide sequence, it is relatively easy to identify such short stretches of identical nucleoticles in each template and design the primer to go along with those stretches to be sequenced. Of course, it is not necessary to limit oneself to codons per se in selecting such stretches and it is preferred to select different stretches per template to be detected. By using additional primers per template in the fashion as described above, the invention is not only applicable to expression profiling per se (where two of more templates are (semi)-quantitatively detected), but it also increases the sensitivity of pyrosequencing applied to one template only. Of course, it is not any more useful in the detection of SNPs, wherein the crux lies in the detection of nucleotide differences in short nucleotide stretches located in close proximity of a primer used. Further to an increase in sensitivity by the employment of one or more additional primers in step b), sensitivity on RNA-derived DNA templates can be also increased by modification of the pyrosequencing reaction mixture.

A further embodiment includes the addition of not always one nucleotide per reaction step, but the addition of sometimes two or three nucleotides per reaction step at one or more steps of the pyrosequencing reaction. In this case, the number of nucleotides that will be incorporated on a particular template in one step of the pyrosequencing reaction step can be varied in order to provide a further signal increase per reaction step. The intensity of the signal corresponds to the number of incorporated nucleotides on a particular template. This modification of the pyrosequencing reaction implies that: first, significant signals can be obtained over longer stretches of the RNA-derived DNA template following the primer annealing site than in the unmodified reaction; second, the pyrosequencing reaction now can be used as a general finger-printing method on larger parts of a RNA genome providing more information about the relatedness of organisms like viruses, pathogens or expressed genes from higher organisms; third, less detailed sequence information is obtained than in the unmodified reaction which is advantageous in cases where polymorphism in signals is not desired, for instance, if a general test is required for a broader range of species or cultivars that exhibit polymorphisms at the locus on which the test is based; fourth, the signal increase per reaction step increases the overall sensitivity of the pyrosequencing reaction, allowing lower amounts of RNA-derived DNA template to be detected; fifth, selection of primer and template sequences that follow the 3′-end of the primer hybridization sites on two different templates and the design of a nucleotide dispensation scheme allows the specific and quantitative determination of each of the two templates in a single reaction mixture using this modification of the pyrosequencing reaction; sixth, due to sensitivity increase, a more accurate ratio can to be determined between different templates in one reaction mixture. A second modification includes the withdrawal of the apyrase enzyme from the reaction mixture which enables continuous synthesis after the successive addition of each of the four different nucleotides has taken place. In the case apyrase is absent and one or two of the four nucleotides are absent from the reaction mixture, synthesis will be limited to relatively short stretches of RNA-derived DNA template. In this situation, the length and the nucleotide composition of the short stretch following the primers directly relates to the intensity of the observed signal.

Therefore, the design of one or more primers which are complementary to the two different templates in a reaction mixture can be done in such a way that the sequences adjacent to the 3′-end of the primers will provide further discriminative signals between the two templates during the first three successive additions of different nucleotides to the reaction mixture as multiple incorporations of nucleotides take place on each template during the second and third nucleotide addition. After the addition of the fourth nucleotide, full synthesis can start on each template resulting in a strong signal, thus providing an additional reference signal for reaction efficiency or total quantity of RNA-derived templates. These further embodiments of the pyrosequencing reaction as mentioned provide an increase in sensitivity instrumental in obtaining a higher range of detection of RNA-derived templates. The same modifications are also applicable to non-RNA-derived templates.

Clearly, a method as provided herein is also applicable to expression products such as mRNA that derive from different genes. This insight provides exciting possibilities for expression profiling as a whole and is not only limited to the detection of a developmental stage of an organism comprising determining the expression of at least a first gene and a second gene, or fragments thereof, involved in development of the organism. Other applications include the detection or determination of the physiological stage the organism is in, its reactivity towards disease or environmental conditions, and so on.

Other examples are a method according to the invention wherein the analogue comprises deoxyadenosine athiotriphospate (dATPaS). Applying a method according to the invention is particularly useful when the first gene and the second gene are variably expressed, especially during development or after having been subjected to environmental stimuli, in this way, changes in time (and thus quality) are easily monitored. As said, a likely option for application of a method according to the application is in the field of plants, however, other organisms are by no means excluded, considering that exactly the same technology serves all needs. The nature and regulation of various processes in such organisms can be determined in detail by combining all available data into a biochemical model. For plants, this is, for example, useful for determining flower wilting, fruit ripening and leaf senescence. This will undoubtedly lead to a better understanding of the ripening process, it will provide the biomarkers for the diagnostic test and it will provide the tools for marker-assisted and molecular breeding towards longer vase life and increased stress resistance. Data generated through the above genomics and bio-informatics approach is directly coupled with a practical application. It is expected that this will result in an intensified interest in the use of biotechnology, not only for agricultural and horticultural problems.

A practical benefit will be that an objective high-throughput automated test system for quality and stage determination in plant products will become available. For ornamentals, but also for other crops or plant products, there are no objective and reliable methods for the determination of internal quality or physiological stage available until now. From a technical point of view, this is very important, as it modifies the use of existing technology for rapid analysis of DNA samples to technology capable of expression analysis. Semi-automated equipment for high-throughput analysis of RNA is not yet available, not even in the medical field, and, in particular, not where a method according to the invention allows step b) and/or a) to be performed in one reaction vessel. The use of semi-automated high-throughput testing facilities will make it possible to optimize transport chains, to certify batches and to perform tracking and tracing. The technology developed herein is of use in all sectors of the horticultural or agricultural industry where fresh products are harvested, handled, transported and stored.

### BRIEF DESCRIPTION OF THE DRAWINGS

FIG. 1A: Light emission profile (pyrogram) of a DNA polymerization reaction carried out in single steps by adding one specific nucleotide at a time. The reaction included one PCR fragment comprising a part of the BGL-gene as a template and one sequencing primer. The order of nucleotide additions is indicated along the y-axis. Arrows indicate the signals which are due to internal folding of the sequencing primer. Peak heights values presented below the figure were used as a quantitative measure for the number of nucleotides reacting at each single step.

FIG. 1B: As in FIG. 1A but now the reaction was performed on a PCR fragment comprising the PRP gene.

FIG. 1C: As in FIG. 1A but now the reaction was performed on a PCR fragment comprising the THA gene.

FIG. 1D: As in FIG. 1A but now the reaction was performed on a PCR fragment comprising the RPL10 gene.

FIG. 1E: As in FIG. 1A but now the reaction was performed on a PCR fragment comprising the RPL12 gene.

FIG. 2A: Light emission profile (pyrogram) of a DNA polymerization reaction carried out in single steps by adding one specific nucleotide at a time. The reaction includes two different PCR fragments, one comprising a part of the THA gene and one comprising a part of the RPL10 gene. One sequence primer annealing to the THA gene fragment and one primer annealing to the RPL10 fragment were added. The order of nucleotide additions is indicated along the y-axis. Peak heights were used as a quantitative measure for the number of nucleotides reacting at each single step. The ratio between the two PCR fragments was calculated from the G 4-mer and C- and T-dimers signals.

FIG. 2B: Light emission profile (pyrogram) of a DNA polymerization reaction carried out in single steps by adding one specific nucleotide at a time. The reaction includes two different PCR fragments, one comprising a part of the PRP gene and one comprising a part of the RPL12 gene. One sequence primer annealing to the PRP gene fragment and one primer annealing to the RPL12 fragment were added. The order of nucleotide additions is indicated along the y-axis. Peak heights were used as a quantitative measure for the number of nucleotides reacting at each single step. The ratio between the two PCR fragments was calculated from the G- and A-dimers signals.

FIG. 2C: Light emission profile (pyrogram) of a DNA polymerization reaction carried out in single steps by adding one specific nucleotide at a time. The reaction includes two different PCR fragments, one comprising a part of the RPL12 gene and one comprising a part of the BGL gene. One sequence primer annealing to the RPL12 gene fragment and one primer annealing to the BGL fragment were added. The order of nucleotide additions is indicated along the y-axis. Peak heights were used as a quantitative measure for the number of nucleotides reacting at each single step. The ratio between the two PCR fragments was calculated from the G-dimers and G- and T-monomers signals.

### DETAILED DESCRIPTION

### EXAMPLES

Quantification of the expression of different genes involved in the ripeness of cucumber using Pyrosequencing.

Primer Design

Primers were designed to make it possible to compare a double incorporation (BGL, PRP vs. RPL12) or a tetra incorporation (THA vs. RPL10) of the same nucleotide (G). The idea was to improve the detectability of a mRNA from a gene with relatively low expression and the two set-ups (2G vs. 4G) was done in order to test and enhance the sensitivity. Moreover, the design for BGL, PRP and RPL12 allows for a triplex detection since nucleotide incorporations from each fragment can be separated according to the nucleotide 3′ of the primer that precedes double G used for quantification. By placing a primer next to a double G on a fourth fragment, it would also be possible to design a tetraplex. Below, the sequencing primers are highlighted and the nucleotides used for quantitative comparison are boxed and underlined.

[image]

Materials and Methods

Template:

Total RNA isolated from plant tissue was converted into cDNA in a oligodT-primed reverse transcriptase reaction. Precipitated RT-PCR products were used. Samples were dissolved in MQ-H₂O according to the strength of the band on the gel, as follows.

| | Volume MQ-H₂O |
|---|---|
| Sample no. | (μl) |
| 1. BGL | 90 |
| 2. PRP | 50 |
| 3. THA | 90 |
| 4. RPL10 | 90 |
| 5. RPL12 | 50 |
| 6. RPL10 + THA | 90 |
| 7. RPL12 + PRP | 50 |
| 8. RPL12 + BLC | 90 |

Sample Preparation and Pyrosequencing:

The sample preparation was carried out according to the Pyrosequencing protocol. Immobilizations were done using 20 μl of the dissolved PCR product together with 10 μl Dynabeads mixed with 30 μl binding buffer. Annealing was done with 15 pmol sequencing primer. Pyrosequencing and subsequent evaluation was done using the PSQ™ 96 SNP Software v. 1.2 AQ.

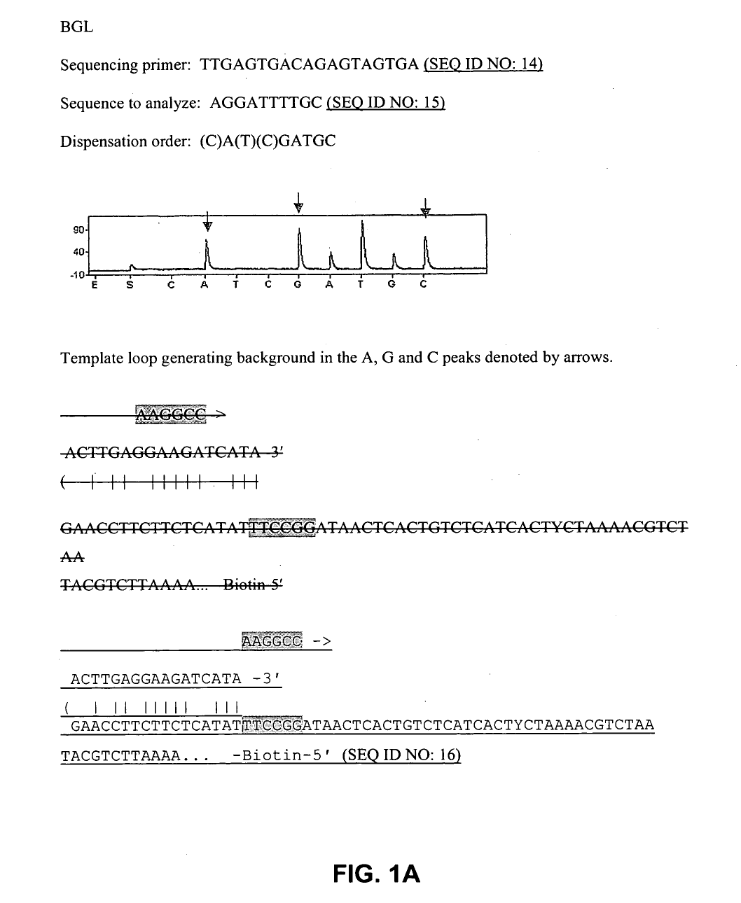

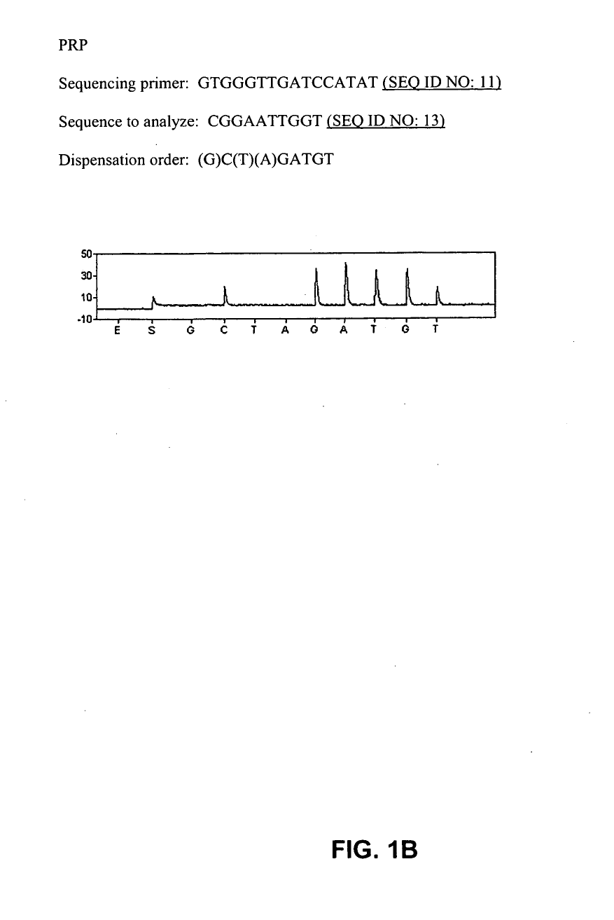

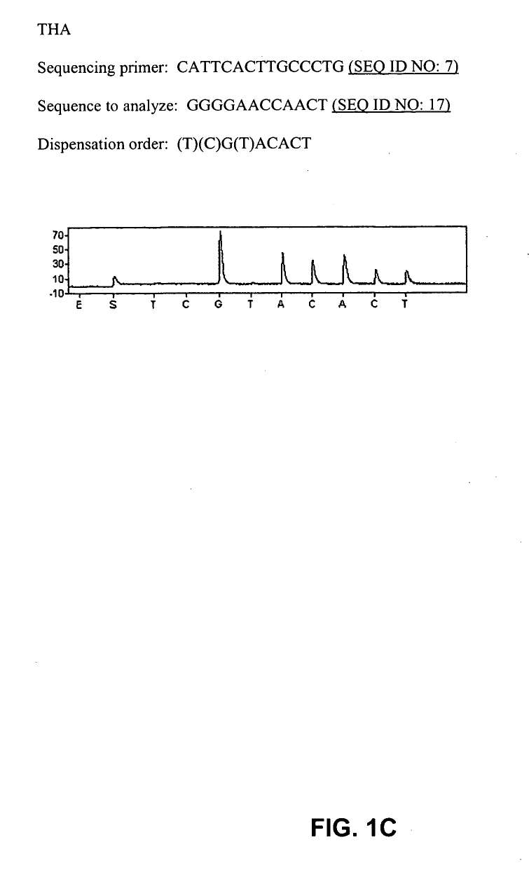

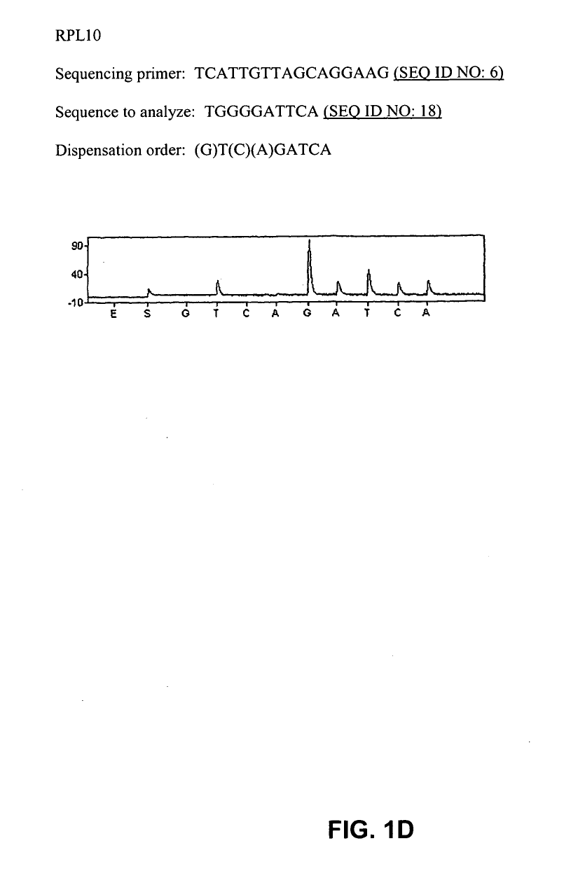

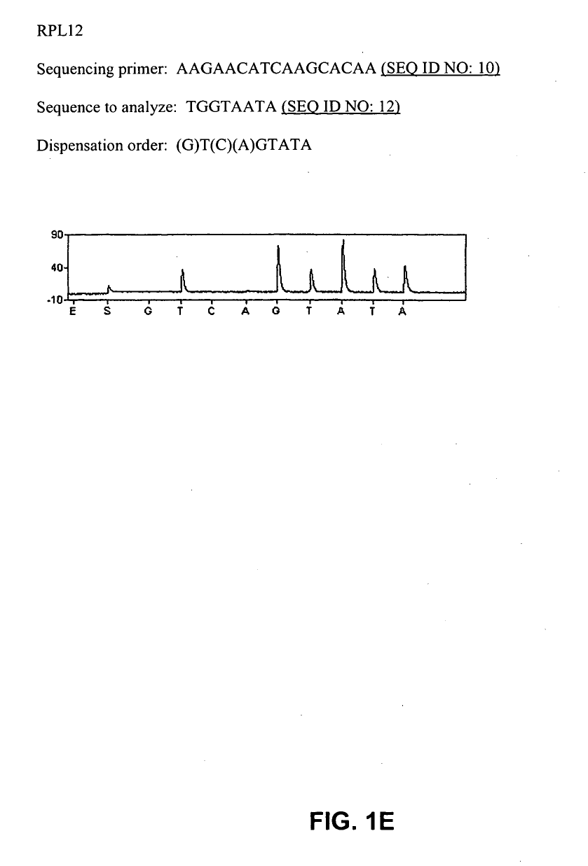

The pyrograms for the runs with only one template (BGL, PRP, THA, RPL10 and RPL12) are presented in FIG. 1. The sequences achieved matches the expected sequences (sequence to analyze) well, except for the BGL (BLC) sequence which is influenced by background generated from extension of the 3′-end of the template, looping back to itself and generating a priming site for the polymerase (see below).

Relative Quantification

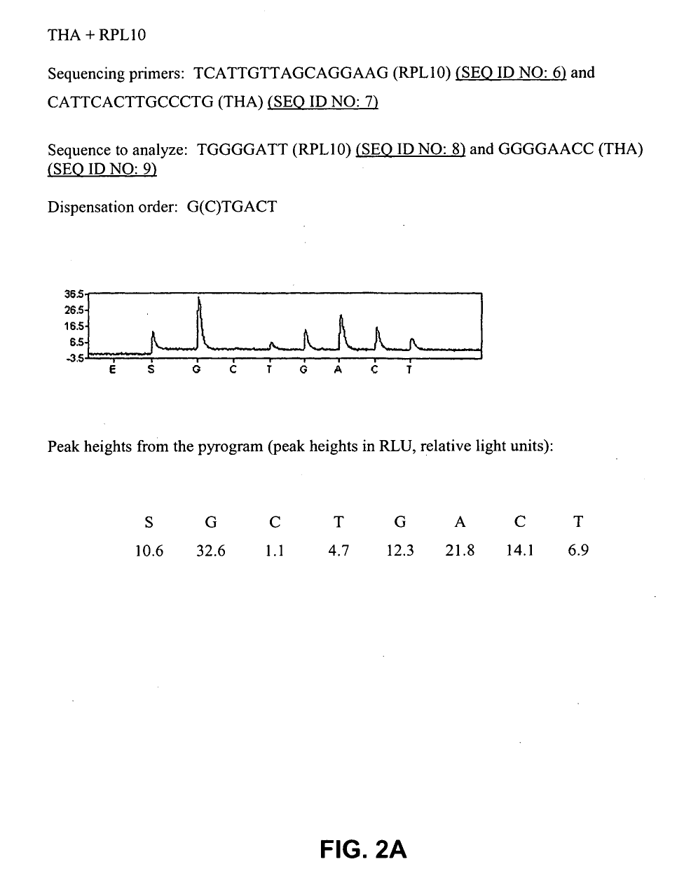

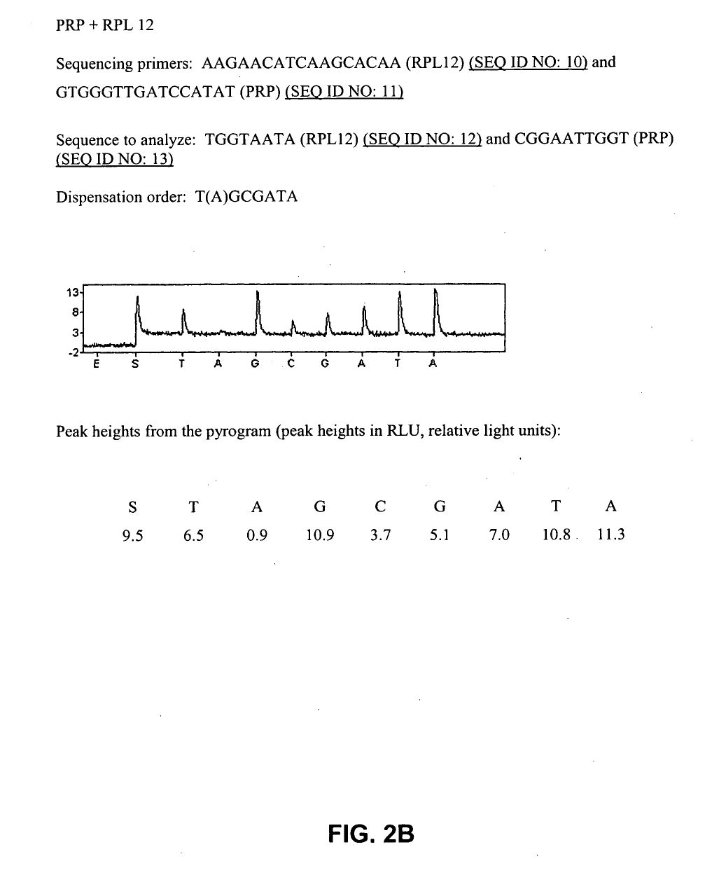

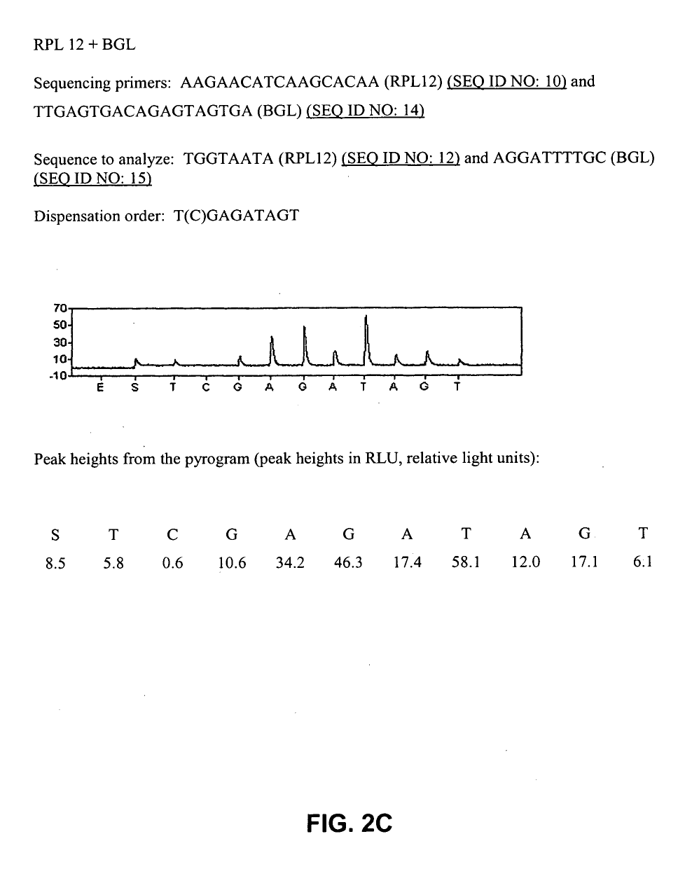

To be able to determine the relative quantity of each template in the samples with mixed templates, dispensation orders were created that made it possible to achieve isolated peaks from each fragment (see FIG. 2). For comparison and additional control, other template-specific peaks were also compared. These calculations are shown in italics.

| | |
|---|---|
| Sequencing primers: | TCATTGTTAGCAGGAAG (RPL10) and |
| | CATTCACTTGCCCTG (THA) |
| Sequence to analyze: | TGGGGATT (RPL10) and GGGGAACC |
| | (THA) |
| Dispensation order: | G(C)TGACT |

With this dispensation order, it is possible to distinguish between peaks from RPL10 and THA. The first G-peak corresponds to the 4-mer in the THA fragment and the second G-peak corresponds to the 4-mer in the RPL10 fragment.

In a similar fashion, the last C-peak corresponds to the C-dimer in the THA fragment and the last T-peak corresponds to the T-dimer in the RPL10 fragment.

Calculation of frequencies is done for both the G 4-mer and the C- and T-dimers.

G 4-mer: % RPL10=(peak height RPL10/(Peak height THA+Peak height RPL10)=12.3 RLU/(32.6 RLU+12.3 RLU)=27%

C- and T-dimers: % RPL10=(peak height RPL10/(Peak height THA+Peak height RPL10)=6.9 RLU/(14.1 RLU+6.9 RLU)=33%

% THA=73 %

% RPL10=27%

PRP+RPL12

| | |
|---|---|
| Sequencing primers: | AAGAACATCAAGCACAA (RPL12) and |
| | GTGGGTTGATCCATAT (PRP) |
| Sequence to analyze: | TGGTAATA (RPL12) and |
| | CGGAATTGGT (PRP) |
| Dispensation order: | T(A)GCGATA |

With this dispensation order, it is possible to distinguish between peaks from RPL12 and PRP. The first G-peak corresponds to the dimer in the RPL12 fragment and the second G-peak corresponds to the dimer in the PRP fragment.

In a similar fashion, the second A-peak corresponds to the A-dimer in the PRP fragment and the last A-peak corresponds to the A-dimer in the RPL12 fragment. Also, the first T-peak corresponds to a monomer from RPL12 and the C-peak to a monomer from PEP.

Calculation of frequencies is done for both the G- and the A-dimers.

G-dimers: % PRP=(peak height PRP/(Peak height RPL12+Peak height PRP)=5.1 RLU/(10.9 RLU+5.1 RLU)=32%

A-dimers: % PRP=(peak height PRP/(Peak height RPL12+Peak height PRP)=7.0 RLU/(11.3 RLU+7.0 RLU)=38%

T- and C-monomers: % PRP=(peak height PRP/(Peak height RPL12+Peak height PRP)=3.7 RLU/(6.5 RLU+3.7 RLU)=36%

% PRP=32%

% RPL12=68%

RPL12+BGL

| | |
|---|---|
| Sequencing primers: | AAGAACATCAAGCACAA (RPL12) and |
| | TTGAGTGACAGAGTAGTGA (BGL) |
| Sequence to analyze: | TGGTAATA (RPL12) and |
| | AGGATTTTGC (BGL) |
| Dispensation order: | T(C)GAGATAGT |

Since there was template background generated from the BGL fragment, the G-peak intended for quantification has to be omitted since the background will add to the peak height and generate a false result. The intention was to use the first G-peak which corresponds to the G-dimer in the RPL12 fragment and the second G-peak that corresponds to the G-dimer in the BGL fragment. Instead peaks that should be unaffected by the background have been used for a preliminary quantification. For this, the last G-peak corresponding to the G-monomer in the BGL fragment and the last T-peak corresponding to the T-monomer in the RPL12 fragment were compared, as well as the first T corresponding to a monomer from RPL12 and the second A corresponding to a monomer from BGL.

Calculation of frequencies is done for both the G-dimers and the G- and T-monomers.

G-dimers: % BGL=(peak height BGL/(Peak height RPL12+Peak height BGL)=46.3 RLU/(10.6 RLU+46.3 RLU)=81%

G- and T-monomers: % BGL=(peak height BGL/(Peak height RPL12+Peak height BGL)=17.1 RLU/(6.1 RLU+17.1 RLU)=74%

T- and A-monomers: % BGL=(peak height BGL/(Peak height RPL12+Peak height BGL)=17.4 RLU/(5.8 RLU+17.4 RLU)=75%

% BGL Average: (75+74)/2=74%

% RPL12=26%

The results show that it is possible to generate template-specific peaks that can be used for relative comparison of different template levels. Using homopolymeric stretches enhances the sensitivity and makes it possible to determine transcripts expressed at low levels vs. those that are well expressed. The results obtained from Pyrosequencing seem to be in agreement with the relative abundance of the templates as deduced from EtBr-stained fragments in an agarose gel. However, from the gel picture there seems to be larger differences in expression levels than shown by the Pyrosequencing data. One possible explanation is that fragments of different lengths bind different amounts of EtBr per molecule. Moreover, staining in a gel might not be uniform throughout the gel.

In order to adjust the BGL assay, the forward PCR primer can be redesigned or just extended in its 5′-end in order to add a couple of mismatching bases.

What is claimed is:

1. A method for determining a developmental or physiological stage of an organism comprising determining the expression of at least a first gene and a second gene, or gene fragment, said method comprising:
providing at least a first nucleic acid template derived from said first gene and a second nucleic acid template derived from said second gene
hybridizing at least one first primer to said first template and at least one second primer to said second template; and
determining binding of said primers to said templates in one reaction vessel.

2. The method according to claim 1, wherein determining binding of said primers to said templates comprises a sequencing step.

3. The method according to claim 2, wherein said sequencing step comprises sequencing amplified DNA.

4. The method according to claim 3, wherein said amplified DNA comprises PCR-amplified DNA.

5. The method according to claim 2, wherein a primer comprises a sequencing primer.

6. The method according to claim 5, wherein said primer can initiate a sequencing reaction carried out by a DNA polymerase.

7. The method according to claim 6, wherein said DNA polymerase is an RNA dependent DNA polymerase.

8. The method according to claim 6, wherein a dATP or ddATP analogue is used which is capable of acting as a substrate for said DNA polymerase, but incapable of acting as a substrate for a pyrophosphate detection enzyme.

9. The method according to claim 8, wherein said analogue comprises deoxyadenosine thio triphosphate (dATPaS).

10. The method according to claim 1, wherein said first gene and said second gene are variably expressed during said development.

11. The method according to claim 1, wherein said organism comprises a plant.

12. The method according to claim 1, wherein hybridizing at least one first primer to said first template and at least one second primer to said second template is performed in one reaction vessel.

13. The method according to claim 1, wherein providing at least a first nucleic acid template derived from said first gene and a second nucleic acid template derived from said second gene is performed in one reaction vessel.

14. The method according to claim 1, further comprising hybridizing at least one additional primer to said first template.

15. The method according to claim 14, wherein said additional primer is selected to hybridize at least in close proximity to a short stretch of identical nucleotides on said first template.

16. The method according to claim 1, wherein hybridizing at least one first primer to said first template and at least one second primer to said second template further comprises hybridizing at least one additional primer to said second template.

17. A method according to claim 16 wherein said additional primer is selected to hybridize at least in close proximity to a relative short stretch of identical nucleotides on said second template.
